(19) United States
(12) Patent Application Publication
(10) Pub. No.: US 2026/0108199 A1
(43) Pub. Date: Apr. 23, 2026

(54) METHOD AND SYSTEM FOR DETECTING NEURODEGENERATIVE ABNORMALITY

(71) Applicant: NEUROVIGIL, INC., Moffett Field, CA (US)

(72) Inventor: Philip LOW, Moffett Field, CA (US)

(73) Assignee: NEUROVIGIL, INC., Moffett Field, CA (US)

(21) Appl. No.: 19/423,950

(22) Filed: Dec. 17, 2025

Related U.S. Application Data
(63) Continuation of application No. PCT/US2024/038397, filed on Jul. 17, 2024.
(60) Provisional application No. 63/527,195, filed on Jul. 17, 2023.

(51) Int. Cl. A61B 5/372; A61B 5/257; A61B 5/291

(52) U.S. Cl. CPC A61B 5/372; A61B 5/257; A61B 5/291

(57) ABSTRACT

The present disclosure relates to a method and system for acquiring and analyzing physiological signals to detect neurodegenerative abnormality in a brain of a subject. A wearable wireless device together with one or more cluster of electrodes can be used to record the physiological signals for each hemisphere of the brain of the subject. For each hemisphere and for each of the one or more cluster of electrodes may include at least one active electrode. The physiological signals for each hemisphere may be separately processed that may include denoising, artifacts removal, segmentation, normalization, transformation, and/or feature extraction. The physiological signals of a first hemisphere and the physiological signals of a second hemisphere of the brain of the subject can be compared to quantify asymmetry across hemispheres and to identify neurodegenerative abnormality.

### CROSS-REFERENCE TO RELATED APPLICATIONS

This application is a continuation of International Patent Application Number PCT/US2024/038397 filed on Jul. 17, 2024, which claims priority to U.S. Provisional Application No. 63/527,195, filed on Jul. 17, 2023. The entire disclosures of the aforementioned applications are incorporated by reference herein in their entireties for all purposes.

### BACKGROUND

Time is of the essence when treating neurodegenerative abnormalities. For example, common treatments for stroke include administering a tissue plasminogen activator (which dissolves blood clots to increase blood flow) or removing a clot using an endovascular procedure. However, if these treatments are not delivered quickly, neurons will have already died due to lack of blood supply. As another example, when a person has experienced a traumatic brain injury (e.g., due to a fall, vehicle accident, etc.), a “primary brain injury” immediately occurs. However, a “secondary brain injury” can occur days after the primary brain injury. The secondary brain injury can include (for example) free radical overload or a subarachnoid hemorrhage, either of which can cause death of neurons and therefore lead to further (irreversible) brain damage or even death. Treatments that can be provided to reduce the likelihood of the secondary brain injury occurring include anti-seizure drugs, coma-inducing drugs, diuretics, and surgery (to remove a clot, repair a skull fracture, stop a brain bleed, or cut away part of the skull to relieve brain pressure).

One complication is that sometimes it is difficult to determine whether a given subject truly experienced a neurodegenerative event. For example, a subject may present with atypical symptoms, which may result in a medical provider not recognizing the possibility of a stroke or other neurodegenerative abnormality. As another example, initial tests may fail to detect signs of the abnormality. Moreover, there is also a need for improved techniques associated with analysis, monitoring, and/or detection of neurodegenerative abnormalities to generate alerts for a caretaker of a subject with neurodegenerative abnormalities. Further, there is a need for improved techniques associated with the analysis, monitoring, and/or detection of neurodegenerative abnormalities, which reduces time for determining cause and effect of the neurodegenerative abnormalities, thereby improving response time and preparation for physiological implications associated with the neurodegenerative abnormalities.

### SUMMARY

Some embodiments of the present disclosure relate to use of physiological signals of a subject to detect neurodegenerative abnormality in a brain of the subject. A computer-implemented method includes obtaining physiological data of the subject that was collected by a physiological data acquisition assembly that comprises one or more clusters of electrodes. The physiological data acquisition assembly may further include one or more sensing devices that can be utilized to acquire and process signals from the one or more clusters of electrodes. Each cluster of the one or more clusters of electrodes comprises at least an active electrode. Other electrodes in each cluster may include a reference electrode, or a bias electrode.

A first portion of the physiological data may be determined that includes first brain activity data for a first hemisphere of the brain of the subject. Similarly, a second portion of the physiological data may also be determined that includes second brain activity data for a second hemisphere of the brain of the subject. The first brain activity data may comprise feature sets corresponding to different segments or epochs of EEG signals of the first hemisphere. Similarly, the second brain activity data may comprise feature sets corresponding to different segments or epochs of EEG signals of the second hemisphere.

The first portion and the second portion of the physiological data may be compared to detect asymmetry across hemispheres. For example, the feature sets representing the first portion and the second portion of the physiological data may be assessed using a criterion to predict whether there is an asymmetry across hemispheres to be indicative of a neurodegenerative abnormality. In some instances, a multi-dimensional distribution may be generated for each of the first brain activity data and the second brain activity data. The multi-dimensional distribution can be generated for each hemisphere, by projecting the associated feature sets into a multi-dimensional space. Afterwards, the multi-dimensional distribution corresponding to the first brain activity data can be compared with the multi-dimensional distribution corresponding to the second brain activity data using a statistical technique. The statistical techniques may include Kolmogorov-Smirnov test, t-test or Wilcoxon signed-rank test. Statistical analysis may generate a p-value that can be compared with a p-value threshold to predict neurodegenerative abnormality.

In some embodiments, after the feature sets are projected into the multi-dimensional space, an inter-hemisphere distance metric can be calculated based on distances between points from different hemispheres. For instance, the inter-hemisphere distance metric can be computed based on the first portion and the second portion of the physiological data. Moreover, an intra-hemisphere distance metric may be computed for each of the first portion and the second portion of the physiological data. A composite score may be generated based on the inter-hemisphere distance metric and the intra-hemisphere distance metric for each the first portion and the second portion of the physiological data.

In some other embodiments, the comparison of the first portion and the second portion of the physiological data may be based on clustering. A clustering technique may be performed using the first brain activity data and the second brain activity data. A first set of clusters can be generated by using the clustering technique on the first brain activity data. Similarly, a second set of clusters can be generated by using the clustering technique on the second brain activity data. The first set of clusters may be compared with the second set of clusters based on a distribution of feature sets of the first brain activity data and distribution of feature sets of the second brain activity data. Clustering may group similar feature vectors together, thereby identifying patterns and similarities within and between the first hemisphere and the second hemisphere. To quantify asymmetry between the first set of clusters and the second set of clusters, a probability value or score can be derived based on how dissimilar the clusters are between the first hemisphere and the second hemisphere.

An output may be generated based on the comparison of the first portion and the second portion of the physiological data. The output may include the p-value, the probability value, or the composite score. The output may indicate that the subject has an increased likelihood of having a tissue abnormality or a neurological disease. The neurological disease may include a stroke and/or the tissue abnormality may include a brain tumor. The output may include an alert that further investigation of treatment for a neurodegenerative anomaly may be prudent. The output and/or the alert may be presented on a device (e.g., smartphone, tablet, laptop etc.) of the subject or a caregiver or may be transmitted to another device or remote device.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an example overview of a system to detect neurodegenerative abnormality in accordance with some embodiments of the present disclosure.

FIG. 2 shows an exemplary placement of an adhesive film, electrodes, and the sensing device on a subject forehead in accordance with some embodiments of the present disclosure.

FIG. 3 illustrates exemplary pipelines to process left and right hemisphere EEG data for a prediction model to generate a neurodegenerative risk score of the subject.

FIG. 4 shows an exemplary implementation of the prediction model of FIG. 3 by using statistical analysis on brain activity data of left and right hemispheres.

FIG. 5 shows another exemplary implementation of the prediction model of FIG. 3 by leveraging clustering techniques on the brain activity data of left and right hemispheres.

FIG. 6 shows another exemplary implementation of the prediction model of FIG. 3 by utilizing distance metrics to generate the neurodegenerative risk score of the subject.

FIG. 7 shows an example flowchart of a system detecting neurodegenerative abnormality in the subject in accordance with some embodiments of the present disclosure.

FIG. 8 shows an example illustration of a computer system in which various embodiments of the present disclosure may be implemented.

### DETAILED DESCRIPTION

Disclosed embodiments of the present disclosure relate to a method and system for analyzing physiological signals and detecting a neurodegenerative abnormality in one or more tissues of a subject. The detected neurodegenerative abnormality may indicate a neurological disease associated with the subject. The detected neurological disease that is associated with the subject includes but is not limited to a stroke, neurotraumatic brain injury, aneurysm, tremor, spasm, epileptic seizure, tumor, edema, and the like, in or around the brain of the subject. According to some embodiments, a technical solution is provided in the present disclosure to a technical problem of detecting a neurodegenerative abnormality in an accurate and quick manner.

In some instances, the neurodegenerative abnormality detection system includes a physiological data acquisition assembly that is configured to obtain physiological data associated with the brain of the subject. In one embodiment, the physiological data is collected upon instructing the subject to perform a particular task (e.g., thinking about how to spell a particular word, visualizing a particular object, writing a particular word, etc.). The physiological data acquisition assembly comprises at least one active electrode and a reference electrode. The physiological data acquisition assembly may further include a ground electrode. Each of the electrodes may be a dry electrode or a wet electrode. Each of one, more or all of the electrodes may include an EEG electrode configured to detect electrical signals. Additionally or alternatively, the physiological acquisition assembly may include electromyography (EMG) electrodes, electrooculography (EOG) electrodes, magnetoencephalography (MEG) electrodes, and the like. Alternatively, the physiological data acquisition assembly may correspond to an invasive electroencephalography (iEEG) device, a functional magnetic resonance imaging (fMRI) device, and the like. The physiological data acquisition assembly may include one or more sensors, such as an accelerometer and/or microphone.

The physiological data acquisition may further include a transmitter and potentially a receiver (which may be a single transceiver). The transmitter can be configured to communicate data corresponding to signals recorded by the electrodes to a computing device that is part of the neurodegenerative abnormality detection system. The computing device may be a device operated by the subject, by a medical provider associated with treating the subject, or an entity facilitating medical monitoring or treatment for the subject. Such communication can occur using any of a variety of commercially available protocols, such as a wireless network, including a short-range connection (e.g., a Bluetooth, Bluetooth low energy (BTLE), or ultra-wideband connection) or over a WiFi network, such as the Internet, etc. In some instances, a receiver is configured to receive an instruction or request from a computing device, such as an instruction to begin recording signals or a request to send data to the computing device.

The physiological data acquisition assembly may include one or more adhesive films, and each electrode may be adhered to a corresponding film. The adhesive film(s) may cover some or all of a surface of at least part of the physiological data acquisition assembly. For example, one or more adhesive films (which may include one or more dots, strips, lines or planes of adhesive material) may be positioned across or near part or all of one or more edges of the physiological data acquisition assembly, along or near part or all of a perimeter of the physiological data acquisition assembly or across part or all of a layer of the physiological data acquisition assembly. In some instances, the physiological data acquisition assembly includes a top layer that includes the adhesive film, where the top layer is top relative to a bottom layer of the physiological data acquisition assembly that is to be positioned on human skin. The adhesive film may be (for example) configured to be reusable, semi-permanently attached and/or permanently attached to other parts of the physiological data acquisition assembly. The adhesive film may be provided separately relative to other components of the physiological data acquisition assembly. The adhesive film may be configured to wrap over other components of the physiological data acquisition assembly or to be positioned between other components of the physiological data acquisition assembly and skin of a subject. It will be appreciated that part or all of the physiological data acquisition assembly may be disposable, reusable and/or semi-reusable. For example, an adhesive film may be reusable for period of time until the adhesiveness degrades to a point that the physiological data acquisition assembly is not beinig reliably held in place. As another example, a first part of the physiological data acquisition assembly may be disposable (e.g., part or all of an adhesive film) and a second part of the physiological data acquisition assembly (e.g., a processing component) may be reusable. In various circumstances, one or electrodes may be reusable or disposable. In some instances, two or more active electrodes are adhered to a single film. In some instances, each of two or more active electrodes are adhered to a different film (where the different films are not connected). The physiological data acquisition assembly, a film, and/or each film may include a transmitter and/or transceiver), which may include a cellular transceiver and/or transmitter. In some instances, the physiological data acquisition assembly may include a wearable component in addition to or instead the one or more adhesive films. The wearable component may include one or more straps, one or more bands, or a cap and may have receiving components (e.g., an opening to receive a patch or an electrode). The wearable component can facilitate ensuring that the electrodes and/or films are positioned at target positions on a subject. In addition, instructions may be provided to a subject to indicate where electrodes and/or films are to be placed. For example, a drawing or photograph may be provided that show where each of one or more films are to be adhered on a subject's head (e.g., with a first film on a left side of a forehead and a second film on a right side of a forehead or with an elongated film positioned across a forehead). In some instances, all the electrodes and/or the physiological data acquisition assembly itself can fit into a single adhesive bandage, part or all of which, depending on the embodiment, can be disposable.

The physiological data acquisition assembly may include a processing component that may perform initial processing using the signals recorded by the electrodes. Such processing may occur using execution of software code and/or using hardware elements. The initial processing may include amplification of the signals recorded by the electrodes, determining a differential signal, applying a filter (e.g., to remove signals around 50-60 Hz or to focus on frequency bands of interest), and/or down sampling the signals. A differential signal may be determined by subtracting a signal from one electrode. For example, a signal from a reference electrode may be subtracted from a signal from an active electrode or a signal from a first active electrode may be subtracted from a signal from a second active electrode.

In some instances, one or more initial processing actions may instead or additionally be performed at the computing device to which the signals are transmitted. The computing device may include a mobile device (e.g., a smart phone), a tablet, a laptop, a desktop computer, a computer server, and the like. In some instances, some or all processing actions disclosed herein are performed within the physiological data acquisition assembly itself.

In some instance, examples of functions performed by the computing system may include but are not limited to determining a portion of the physiological data including brain activity data for a first hemisphere of the brain of the subject, determining a portion of the physiological data including brain activity data for a second hemisphere of the brain of the subject, comparing the physiological data including the brain activity data for the first hemisphere of the brain with the physiological data including the brain activity data for the second hemisphere of the brain of the subject, generating a result of the comparison indicating a neurodegenerative abnormality in the brain of the subject, and the like, as described in further detail below.

In some instances, the first hemisphere of the brain of the subject relates to one of a left hemisphere or a right hemisphere of the brain of the subject, where each of the left hemisphere and the right hemisphere controls muscles in the right and the left side of the subject body, respectively. The second hemisphere of the brain of the subject relates to one of a left hemisphere or a right hemisphere of the brain of the subject, other than the first hemisphere of the brain. In some instances, the signals are transmitted with metadata or in an order so as to indicate which signals correspond to electrodes that were to be placed on contralateral sides of a subject. In some instances, the computing device associates various signals from active electrodes with different sides of a subject but does not specifically determine or predict which signals specifically correspond to a “left” side or hemisphere or a “right” side or hemisphere.

In some instances, EEG signals may be examined in time series increments called epochs. The epochs can be segmented into different sections using a scanning window, where the scanning window defines different sections of the time series increment. The scanning window can move via a sliding window (where sections of the sliding window have overlapping time series sequences) or via a jumping window (where sections are non-overlapping). Alternatively An epoch can alternatively span an entire time series, for example.

For each segment and each hemisphere, the signal corresponding to the hemisphere (e.g., which can include a differential signal and/or a preprocessed signal) can be transformed from the time domain to the frequency domain. In some instances, multiple transformations can be performed (e.g., in accordance with the SPEARS algorithm, which is disclosed in U.S. application Ser. No. 11/431,425, filed on May 9, 2006, which is hereby incorporated by reference in its entirety for all purposes). One or more normalizations may be applied in the time domain and/or in a frequency domain. For each segment and each hemisphere, one or more features may be defined, which may include or be based on the power (or normalized power) in the transformed signal at each of one or more frequency bands. The frequency bands may include a band corresponding to Delta, Gamma, Alpha, or Beta frequencies or any other frequency range. A feature may include a weighted average of power across multiple frequency bands (e.g., where the weights are determined by a component analysis, neural network, etc. that has been configured or trained to differentiate signals from subjects with a neurodegenerative abnormality versus healthy subjects). In some instances, the weights are determined based on one or more signals previously collected from the subject. The feature may be used to detect whether there is neurodegeneration and/or an extent of neurodegeneration by (for example) assessing the feature in view of data previously collected from the subject (e.g., one or more features derived using one or more previously collected signals) and/or data collected from other subjects (e.g., at least some of which experienced a neurodegenerative abnormality).

A feature may include a statistic that is determined based on one or more power values or weighted power values. For example, a feature may include a maximum or minimum power (or normalized power) in a spectrum corresponding to a segment, a standard deviation (across frequency bands) of power, etc. As another example, a feature may include a standard deviation of power values associated with a given frequency band (or weighted power values) across segments. As yet another example, a feature may include a z-score, which can include a normalized unit that reflects the amount of power in the signal, relative to the average of the signal. The z-scores can be converted into mean deviation form, by subtracting the mean from each score. The scores can then be normalized relative to standard deviation. All of the z scored normalized units can have standard deviations that are equal to unity.

As one illustration, features may be defined to include normalized power in a low frequency band (e.g., Alpha band), normalized power in a high frequency band (e.g., Gamma band), standard deviation of normalized power values across frequency bands in an epoch, a maximum normalized power value for the epoch, and the sort. Further features can be defined based on information calculated for each of the one or more epochs corresponding to each of the first hemisphere and the second hemisphere of the brain to create information such as Gamma power/Alpha power, time derivative of Delta, time derivative of Gamma power/Alpha power and the like. Time derivatives can be computed over preceding and successive epochs. After calculating the information, it can then be normalized across the one or more epochs. A variety of data normalization techniques can be conducted including z-scoring and the like. In this way, the higher frequency data may become more clearly visible.

Thus, a variety of features may be calculated for each hemisphere of a subject. Some features may correspond to individual epochs, while others may apply to multiple or all epochs associated with a given time period (e.g., night, day, or set of days). In this case, feature sets associated with individual epochs may be expanded to include each multi-epoch feature that corresponds to a time period overlapping with the epoch.

The feature sets may be assessed using a criterion to predict whether there is a sufficient asymmetry across hemispheres to be indicative of the neurodegenerative abnormality. The criterion may identify a threshold for a score, probability, p-value, metric, such that a corresponding subject-specific calculated value can be compared to threshold to predict whether the subject has or may have a neurodegenerative abnormality. For example, for each hemisphere, a multi-dimensional distribution may be generated using the associated features. A statistical test can then be performed to predict a probability that the two distributions correspond to a same underlying distribution. The statistical test can include generating a p-value, which can then be compared to a p-value threshold. As another example, after the features are projected into a multi-dimensional space, an inter-hemisphere distance metric can be calculated based on distances between points from different hemisphere. Similarly, one or more intra-hemisphere distance metrics can be calculated based on distances between points from a same hemisphere. A score corresponding to a probability of there being an asymmetry caused by a brain abnormality can be defined based on the metrics, and the score can then be compared to a score threshold. As yet another example, a clustering technique may be applied, such that each feature set (corresponding to a given epoch and a given hemisphere) is assigned to a cluster. A probability of there being an asymmetry caused by a brain abnormality can be generated based on characterizing an extent to which various clusters included feature sets associated with different hemispheres (relative to an extent to which various clusters included feature sets associated with same hemispheres), and the probability can then be compared to a probability threshold. It will be appreciated that an asymmetry can be brain state/time dependent and/or can occur in a limited range of frequencies, including a single time/brain/state or frequency.

In one embodiment, the neurodegeneration is measured or overcome in response to a task (spelling with one's mind). In another embodiment, the neurodegeneration is dismissed/assessed in response to a task (spelling in a patient thought to be comatose/vegetative/minimally conscious) or to a stimulus (detection of a neural response associated with surprise when subject is presented with “surprising stimulus” elicited spelling in a subject thought to be comatose, vegetative and/or minimally conscious.

In some instances, a result of processing the feature sets (e.g., a p-value, score based on distances, probability based on clustering) may be output automatically upon being generated. In some instances, the result is selectively output (e.g., upon determining that the criterion is satisfied and/or upon receiving and verifying a request for the same). The result may be output along with other data, such as one or more feature sets (e.g., a first set of feature sets associated with a subset of epochs and a first hemisphere and a second set of feature sets associated with the same subset of epochs and a second hemisphere). The output may include presenting the result on the computing device or transmitting the result to another device. The output may include an alert that further investigation or treatment for a neurodegenerative anomaly may be prudent.

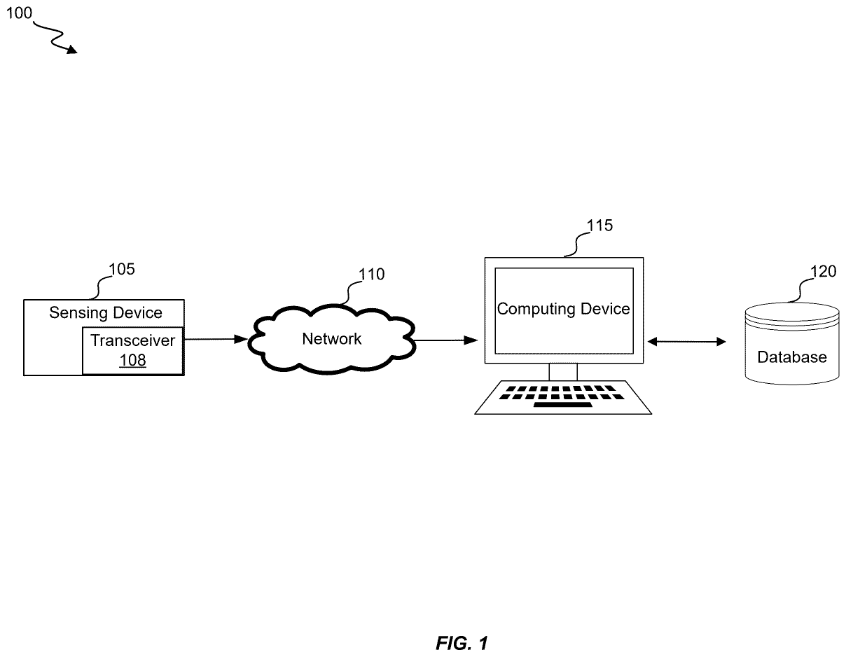

FIG. 1 illustrates an example overview of a system to detect neurodegenerative abnormality in accordance with some embodiments of the present disclosure. Exemplary system 100 comprises a sensing device 105, a network 110, one or more computing devices 115, and one or more databases 120. The sensing device 105 may include a transceiver 108 to communicate with the computing device 115. The sensing device 105 may be connected to the computing device 115 through the network 110. The transceiver 108 can be configured to communicate physiological data recorded by the sensing device 105 to the computing device 115 that is part of the neurodegenerative abnormality detection system.

The computing device 115 may be a device operated by the subject, by a clinician or the medical provider associated with treating the subject, or an entity facilitating medical monitoring or treatment for the subject. The computing device 115 may include a mobile device (e.g., a smart phone), personal digital/data assistants (PDA), a tablet, a laptop, a desktop computer, a computer server, and the like. In some instances, the transceiver 108 and/or the sensing device 105 can be configured to receive an instruction or request from the computing device 115, such as an instruction to begin recording signals or a request to send data to the computing device 115. Moreover, the communication between the sensing device 105 and the computing device 115 can occur using the network 110, which can be a wireless network based on a commercially available communication protocols, for example, Bluetooth, Bluetooth low energy, ultra-wideband connection, or WiFi network, such as the Internet, etc. The network 110 may include, internet, an intranet, a wired LAN (local area network), a wireless LAN (WiLAN), a WAN (wide area network), a MAN (metropolitan area network), a PSTN (public switched telephone network) and other types of communications networks. The network 110 may further include communication devices such as one or more gateways, routers, or bridges. The network 110 may be any type of network familiar to those skilled in the art that can support data communications using any of a variety of available protocols, including without limitation TCP/IP (transmission control protocol/Internet protocol), SNA (systems network architecture), IPX (internet packet exchange), AppleTalk®, and the like.

According to some embodiments, the physiological data acquisition assembly may include the sensing device 105, different types of electrodes, and/or wearable components. The different types of electrodes may include but are not limited to dry electrodes, wet electrodes, self-adhesive conductive electrodes, electrodes with snap connectors, the EEG electrodes, the EMG electrodes, the EOG electrodes, the MEG electrodes, and the like.

In some embodiments, the sensing device 105 is configured to acquire, record, process, and transmit physiological data associated with the brain of the subject. In some instances, the physiological data comprises of electrical activity of the brain that can be recorded using EEG electrodes attached to the scalp or on the forehead of the subject. The sensing device 105 may be connected with at least one active electrode and a reference electrode. The active electrode acts as a primary sensor that detects the electrical activity generated by neuronal firing in the brain. The active electrodes pick up the electrical potentials generated by brain activity and transmit these signals to the sensing device 105 for initial processing (e.g., signal amplification, analog to digital conversion, denoising etc.) and analysis. The reference electrode provides a baseline or common point of comparison for the active electrodes. The sensing device 105 may further include a ground (or bias) electrode. The ground or bias electrode may be (for example) placed behind the ear of the subject or included within a part of the physiological data acquisition assembly that includes at least one active electrode. For example, the ground or bias electrode may be included in a patch that includes one or more active electrodes (e.g., and a reference electrode). The bias electrode functions to stabilize the baseline or electrical potential of an EEG system and reduces noise or interference from external electrical sources. In some instances, the physiological data acquisition assembly may omit the ground or bias electrode and may utilize the reference and active electrodes for data recording. This is due to the reason that modern differential amplifiers can be designed to operate without a dedicated ground electrode by using a virtual ground that can be created internally by the amplifier circuitry.

In some other embodiments, the sensing device 105 may be configured to record and store physiological data in encrypted format and to transmit wirelessly to a remote center or the computing device 115 for further display, storing, processing, and analysis. In another aspect of the present disclosure, the recorded and stored physiological data may be transmitted wirelessly in real-time to the computing device 115 including but not limited to a cellular telephone, smart-phone, tablet, and/or computer. The recorded and stored physiological data may also be transmitted directly to a computer, cellular telephone, smart phone and/or tablet via USB transfer capabilities incorporated within the sensing device 105.

During physiological data acquisition, an initial or pre-amplification may be performed at or near the electrodes to reduce noise. For example, the electrode snap connector assemblies may include a noise reducing or cancelling filter at the electrode connection level to reduce any electrical noise that may be picked up by the lead wires. To further improve the physiological data acquisition process, the sensing device 105 or the electrode snap connector assemblies can be configured to continuously monitor electrode impedance and may include lights indicative of the current status of the integrity of the electrode connection with the scalp or forehead. The sensing device may be comprised of hardware and software components (e.g., firmware or signal processing code) and can be used to perform initial processing. The initial processing may include amplification of the signals recorded by the electrodes, determining a differential signal, applying a filter (e.g., to remove signals around 50-60 Hz or to focus on frequency bands of interest), and/or down sampling the signals. A differential signal may be determined by subtracting a signal from one electrode. For example, a signal from a reference electrode may be subtracted from a signal from an active electrode or a signal from a first active electrode may be subtracted from a signal from a second active electrode.

In some instances, one or more initial processing actions may be performed at the computing device 115 to which the signals are transmitted. For example, functions performed by the computing device 115 may include but are not limited to determining a portion of the physiological data including brain activity data for a first hemisphere of the brain of the subject, determining a portion of the physiological data including brain activity data for a second hemisphere of the brain of the subject, comparing the physiological data including the brain activity data for the first hemisphere of the brain with the physiological data including the brain activity data for the second hemisphere of the brain of the subject, generating a result of the comparison indicating a neurodegenerative abnormality in the brain of the subject, and the like.

It will be appreciated that any processing or computation disclosed herein (e.g., to detect a feature, remove noise, detect a temporal change, compare to a threshold, etc.) may be performed using a differential signal across hemispheres. Alternatively or additionally, such processing may be separately performed for each hemisphere and a comparison across hemispheres may then be performed based on the processing results.

The sensing device 105 may further include a battery power component that can include a rechargeable small form factor, high-capacity battery. The sensing device 115 includes a power supply and recharging circuitry for receiving power through an electrical power cord and AC unit. Charging may alternatively or additionally occur using induction. The electrical power cord is coupled to the sensing device 105 for recharging the small form factor, high-capacity battery through a port, which may be but is not limited to USB, DB-25 or the like. The sensing device 105 includes a power on and off function for preserving the power supply of the small form factor, high-capacity battery when not in use. The sensing device 105 may be configured to control whether a battery or power supply is operational. For example, a battery or power supply may be shut off when the sensing device 105 detects that the physiological data acquisition assembly is not recording a signal and/or is not being worn. The sensing device 115 may also include power on and off indicator lights indicative of the current status of the sensing device 105. In some instances, the sensing device 105 may be recharged through a USB connection to a computer.

The exemplary system 100 may further include the one or more databases 120 for storing and future processing of data (e.g., EEG signals of the subject). The physiological data of the subject may be stored with metadata. The metadata may include subject information, type and placement location of each electrode etc. The one or more databases 120 may be integral to a memory system on the computer or in secondary storage such as a hard disk, floppy disk, optical disk, or other non-volatile mass storage devices. Part or all of the one or more databases 120 and/or memory storage units may be located on the physiological data acquisition assembly. The computing device 115 may be used to execute signal processing techniques or algorithms on the physiological data (previously recorded signals or real-time signals) and to store the results in the one or more databases 120.

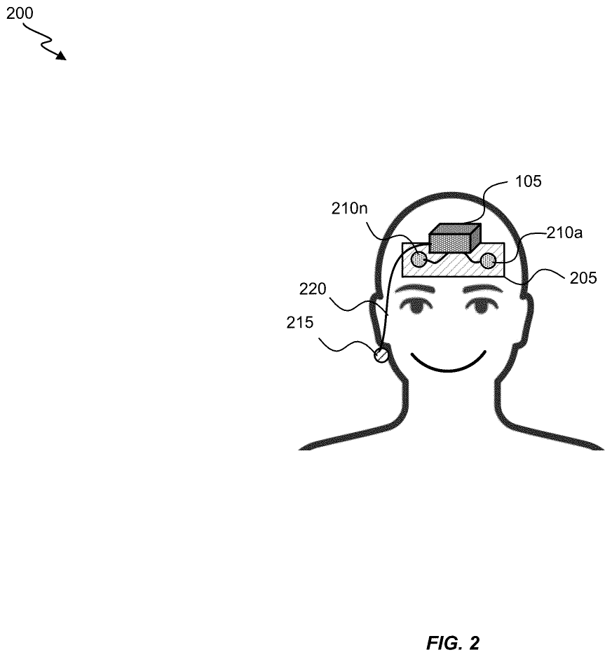

FIG. 2 shows an exemplary placement of an adhesive film 205, electrodes, and the sensing device 105 in accordance with some embodiments of the present disclosure. According to the exemplary placement 200, two or more electrodes 210a-n and the sensing device 105 may be adhered to the adhesive film 205 to capture the left hemisphere and the right hemisphere signals. In some instances, the adhesive film 205 can be a single adhesive film that is attached to the forehead of the subject. The two or more electrodes 210a-n may include the active electrodes and the reference electrodes. Both active and reference electrodes can be placed in close proximity with respect to each other but are not electrically connected. A bias electrode 215 may be attached to the ear (e.g., ear lobe or back side of ear) of the subject using an electrode lead 220. In some other instances, the bias electrode 215 can be skipped and the physiological data acquisition assembly may only include the adhesive film 205 together with the two or more electrodes 210a-n and the sensing device 105.

In some embodiments, the adhesive film 205 may be comprised of stretchable materials. In some instances, the two or more electrodes 210a-n along with connecting wires (or electrode leads 220) may be implemented using a flexible printed circuit board (PCB) and can be attached to the subject using an adhesive material or some type of gel for better signal acquisition. The sensing device 105 may be adhered on the flexible printed circuit board and can be connected to the two or more electrodes 210a-n through PCB traces. In some other instances, both the sensing device 105 and the two or more electrodes 210a-n may be implemented jointly on the flexible PCB that may act as a singular sensor patch. Moreover, the electrode structure (e.g., the number of electrodes or channels, their locations, size etc.) on the flexible PCB can be controlled during the fabrication process.

In some other embodiments, two separate patches may be used to capture the left hemisphere and the right hemisphere data. For example, two modules of the singular sensor patch can be used and may be attached at two different locations such as one on each side of the brain or on the forehead. In some instances, disjoint adhesive films can be used at different locations. Two or more electrodes may be adhered to each of the disjoint adhesive films. Similarly, each adhesive film may be connected with the transceiver 108 or the sensing device 105.

In some instances, the physiological data acquisition assembly may include a wearable component in addition to or instead the adhesive film(s). The wearable component may include one or more straps, one or more bands, or a cap and may have receiving components (e.g., an opening to receive a patch or an electrode). The wearable component may include a helmet, ear piece and/or one or more headphones. The wearable component can facilitate ensuring that the electrodes and/or films are positioned at target positions on a subject. In some cases, the sensing device 105 can be housed in the wearable component such as a head harness, for example, in accordance with the head harness and wireless EEG monitoring system, which is disclosed in U.S. application Ser. No. 17/214,574, filed on Mar. 26, 2021, which is hereby incorporated by reference in its entirety for all purposes. The head harness comprises of straps, and fasteners (e.g., velcro, hook, button etc.) for custom fit, adjustment, and comfort of the subject. The head harness may further include plurality of slots for attaching electrodes or electrodes snap connectors at specific positions. For example, the bias electrode and the reference electrode may be attached behind the left and right ear and the reference electrodes on the forehead of the subject.

In addition, instructions may be provided to the subject that indicate where the electrodes and/or the adhesive films are to be placed. For example, a drawing or photograph may be provided that show where each of one or more adhesive films are to be adhered on a subject's head (e.g., with a first film on a left side of a forehead and a second film on a right side of a forehead or with an elongated film positioned across a forehead).

The physiological data or signals may be acquired during a resting state, sleeping state, or a task-specific state (e.g., eyes closed, thinking task etc.). Typically, the EEG signals from the right hemisphere and the left hemisphere exhibit patterns of synchronization and similar power in different frequency bands particularly during sleeping or resting states for a healthy subject. This phenomenon is also called bilateral symmetry in EEG signals. Bilateral symmetry is a characteristic feature of resting or sleep states, reflecting synchronized brain activity between the two hemispheres. Further, specific signals and frequency intensities are typically highly coherent across hemispheres in healthy subjects during particular sleep and wake stages. Therefore, a statistic that represents a degree of synchronization, correspondence, symmetry, and/or coherence similarity may be used to predict a degree of neurodegeneration.

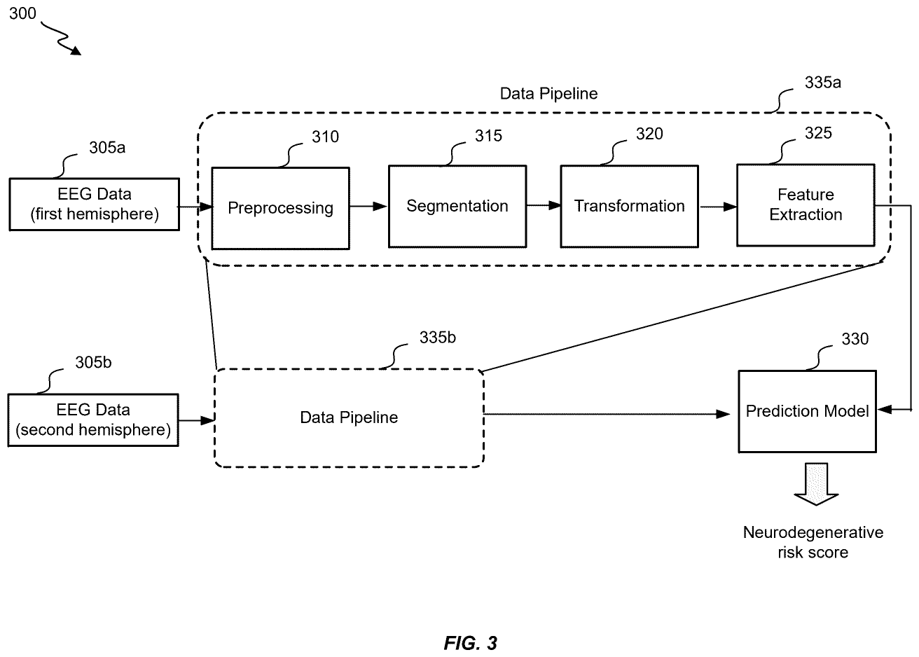

FIG. 3 illustrates exemplary pipelines to process left and right hemisphere EEG data for a prediction model that generates a neurodegenerative risk score (or neurodegenerative abnormality risk score) of the subject. In some instances, EEG data of the first hemisphere 305a of the brain of the subject relates to one of a left hemisphere or a right hemisphere of the brain of the subject. Similarly, EEG data of the second hemisphere 305b of the brain of the subject relates to one of a left hemisphere or a right hemisphere of the brain of the subject, other than the first hemisphere of the brain. In some instances, the sensing device 105 may transmit the EEG signals with metadata or in an order so as to indicate which EEG signals correspond to electrodes that were to be placed on contralateral sides of the subject. In some instances, the computing device 115 may associate various signals from active electrodes with different sides of the subject but does not specifically determine or predict which signals specifically correspond to a “left” side or hemisphere or a “right” side or hemisphere.

After receiving the EEG data 305a-b from the sensing device 105, further processing and analysis can be performed on the computing device 115 as illustrated in FIG. 3. EEG data of the first hemisphere 305a (e.g., left hemisphere) and EEG data of the second hemisphere 305b (e.g., right hemisphere) may be processed using a data pipeline 335a and a data pipeline 335b, respectively. Both data pipelines 335a-b comprise of similar modules such as preprocessing 310, segmentation 315, transformation 320, and feature extraction 325.

The EEG data 305a-b may be processed to remove noise and other signal artifacts at preprocessing 310 module. The preprocessing 310 may involve different types of filters such as band pass, high pass, or low pass to extract particular frequency bands for feature extraction 325. During preprocessing 310, the EEG data 305a-b for each hemisphere may optionally be treated for removing artifacts, where an artifact refers to any part of the EEG data 305a-b that misrepresents the data intended to be received (e.g., movement data in an EEG signal). These artifacts may occur due to e.g., muscle activities such as jaw clenching or head movements causing high-frequency noise, periodic disturbances caused by electrical activity of heart, or other environmental artifacts such as electromagnetic interferences, thereby impacting the accuracy of neurodegenerative abnormality detection. These artifacts can be removed from the EEG data 305a-b, for example, by manually removing i.e., by visually inspecting EEG signals (and/or in parallel observing the subject) and rejecting segments that include large-amplitude fluctuations or sudden changes that are likely to be artifacts or automatically filtering out of the EEG data 305a-b via a filtering (e.g., DC filtering) or data smoothing technique.

The EEG data 305a-b (or signals) for each hemisphere can also be pretreated with component analysis i.e., by decomposing EEG signals into independent components, identifying and removing artifacts based on the spatial and temporal characteristics. EEG artifacts may also be removed by estimating the artifact subspace using methods e.g., principal component analysis (PCA) and projecting the EEG data 305a-b onto orthogonal subspace for artifacts removal. In other instances, template matching may be performed that may identify and remove known artifact patterns by comparing the EEG data 305a-b with predefined templates. Additionally, wavelet transform may be applied that decomposes the EEG data 305a-b into different frequency components using wavelet transform and remove artifacts in specific frequency bands.

After preprocessing 310, segmentation 315 may be performed on the EEG data 305a-b (or EEG signals) that splits the EEG signals (or continuous EEG signals) into multiple time series increments (also referred herein as epochs) of similar or varying lengths. During segmentation 315, the time series increments or epochs may be segmented further into different sections using a scanning window, where the scanning window defines different sections of the time series increment (or epoch). The scanning window can move via a jumping window and resulting sections or segments are non-overlapping. For example, a one-hour epoch or time series increment of a received EEG signal can be scanned or segmented in increments of 1 minute (i.e., a scanning window of 1 minute), thus resulting in 60 disjoint or non-overlapping sections of the one-hour epoch. The scanning window can use a sliding window, where sections (or segments) of the sliding window may have overlapping time series sequences. For example, the one-hour epoch of the received EEG signal can be scanned with a 1-minute scanning window that begins every 30 seconds (i.e., a sliding window of 30 seconds), thus resulting in a 1-minute scanning window that overlaps by 30 seconds. Alternatively, without segmentation 315, an entire time series of the EEG signals may correspond to an epoch.

The segments of the EEG signal for each hemisphere (e.g., which can include a differential EEG signal and/or a preprocessed EEG signal) can be transformed from a time domain to a frequency domain by transformation 320 module. For this purpose, power spectrum may be calculated e.g., by calculating power spectral density of each segment of the EEG signals. The power may be calculated by different techniques such as multi-taper transform, Fourier transform, or wavelet transform. For each segment of the EEG signal and for each hemisphere, one or more normalizations may be applied in the time domain and/or in the frequency domain by transformation 320 module. In some instances, the EEG signal may be adjusted to account for differences in power by performing normalization. For example, normalization may be performed by weighing the spectral power of one or more segments (or time intervals) across time. The normalized power of each segment or time interval at one or more frequencies across time may help determining particular frequency windows for extracting information. Such normalization can reveal low power and statistically significant shifts in power at one or more frequencies bands. The frequency bands may include a band corresponding to Delta, Gamma, Alpha, or Beta frequencies or any other frequency range.

EEG signals may be characterized by different frequency bands, each associated with specific cognitive and physiological states. For example, Delta band that ranges typically around Hz comprising slow waves or frequencies with high amplitudes. Similarly, Theta band that may range approximately around [4-8] Hz, comprises moderate frequencies and amplitude. Alpha band may range approximately around [8-12] Hz and may characterize moderate frequencies with lower amplitudes than Delta and Theta band. Various states such as relaxing, wakefulness or closed eyes may be associated with Alpha band. Additionally, Alpha band may facilitate the transition between wakefulness and sleep. Followed by Alpha, Beta band approximately ranging from [12-30] Hz may be characterized by higher frequency with lower amplitude that may be associated with active thinking, focus, wakefulness, or similar activities. The frequency band with relatively higher frequencies, Gamma, approximately ranging [30-100] Hz may be characterized by high frequencies of EEG signals with low amplitude. Gamma band may be associated with high-level information processing and perception such as rapid eye movement sleep that may be characterized by vivid dreaming and high brain activity resembling wakefulness. By processing these spectral characteristics of spectral bands, brain activity labels (e.g., various sleep stages, resting state, wakefulness etc.) may be assigned to segments of the EEG signals.

Among the frequencies in the signals, any frequency band can be revealed and utilized for further analysis. Feature extraction 325 may be performed on each segment of the EEG signal for each hemisphere. Therefore, for each segment and each hemisphere, one or more features may be defined, which may include or be based on the power (or normalized power) in a transformed signal at each of one or more frequency bands. In some instances, a feature may be generated using signal components corresponding to different frequency bands and/or different epochs, such as a feature corresponding to spectral and/or temporal fragmentation. The one or more features may include a statistic that is determined based on one or more power values or weighted power values. For example, a feature may include a maximum or minimum power (or normalized power) in a spectrum corresponding to a segment, or a standard deviation (across frequency bands) of power, etc. As another example, a feature may include a standard deviation of power values associated with a given frequency band (or weighted power values) across segments. As yet another example, a feature may include a z-score, which can include a normalized unit that reflects the amount of power in the signal, relative to the average of the signal. The z-scores can be converted into mean deviation form, by subtracting the mean from each score. The scores can then be normalized relative to standard deviation. All of the z scored normalized units can have standard deviations that are equal to unity. A feature may include a weighted average of power across multiple frequency bands, for example, the weights can be determined by a component analysis, or a neural network that has been configured or trained to differentiate signals from subjects with a neurodegenerative abnormality versus healthy subjects.

Features may be calculated epoch-wise by using each of the one or more epochs corresponding to each of the first hemisphere and the second hemisphere of the brain. As one illustration, features may be defined to include normalized power in low frequency bands (e.g., Delta band, Theta band, Alpha band), normalized power in a high frequency band (e.g., Gamma band), standard deviation of normalized power values across frequency bands in an epoch, a maximum normalized power value for the epoch, and the like. In addition, derived features can be generated based on the information (or normalized features) calculated for each of the one or more epochs corresponding to each of the first hemisphere and the second hemisphere of the brain. The derived features may include but not limited to Gamma power/Delta power, Gamma power/Alpha power, time derivative of Delta, time derivative of Gamma power/Delta power, time derivative of Gamma power/Alpha power. Time derivatives can be computed over preceding and successive epochs. Afterwards, the derived features can then be normalized across the one or more epochs. A variety of data normalization techniques can be conducted including z-scoring, min-max scaling, quantile transformation, log transformation and other similar techniques. In some instances, normalization is performed by z-scoring that is a statistical technique to standardize the range of independent variables (or features). It may involve transforming the features such that the features have a mean of zero and a standard deviation of one. By applying z-scoring, different derived features of the spectral power data such as Delta power and Gamma power/Delta power may be scaled to a common range, thus eliminating biases.

Thus, a variety of features may be calculated for each hemisphere of a subject. Some features may correspond to individual epochs, while others may apply to multiple or all epochs associated with a given time period (e.g., night, day, or set of days). The computing device 115 may retrieve the historical data of the subject from the database 120 to calculate features for the given time period. In this case, feature sets associated with individual epochs may be expanded to include each multi-epoch feature that corresponds to a time period overlapping with the epoch.

The variety of features that are calculated for each hemisphere of the subject may be fed into a prediction model 330. The prediction model 330 may compare the variety of features corresponding to EEG data of the first hemisphere 305a with the variety of features corresponding to EEG data of the second hemisphere 305b. For example, for each hemisphere, the variety of features or features set obtained from the EEG signals (or processed EEG signals using the data pipelines 335a-b) that corresponds to a particular epoch or a particular time period may be compared to detect neurodegenerative abnormalities. For example, the variety of features or features set generated based on the EEG signals (or processed EEG signals using the data pipelines 335a-b) corresponding to each hemisphere from a particular epoch or a particular time period may be compared to detect neurodegenerative abnormalities. The variety of features or feature sets may be assessed using a criterion to predict whether there is asymmetry across hemispheres to be indicative of a neurodegenerative abnormality. The criterion may identify a threshold for a p-value, a probability, a score, or a metric, such that a corresponding subject-specific calculated value can be compared to threshold to predict whether the subject has or may have a neurodegenerative abnormality. In some instances, multiple variables (e.g., risk scores, multiple risk metrics, multiple probabilities, multiple p-values, etc.) are generated and assessed in accordance with one or more criteria. For example, a single criterion may be defined to be satisfied if any of a set of variables exceeds a corresponding variable-associated threshold. As another example, a set of criteria may be defined such that each criterion is configured to be satisfied if a variable exceeds a variable-specific threshold dynamically determined based on a subject-specific history of variables.

In some instances, an analysis may be performed to assess one or more variables to predict a location, type and/or severity of a neurodegenerative abnormality. For example, multiple risk scores can be generated, such as one for the likelihood of any neurodegeneration, the others for the likelihoods of specific types of degeneration, etc. In some embodiments, a variable is based on the dissimilarity between the hemispheres or across time in the same subject. In some embodiments, a variable is based on similarity between the features, and the variable may be predictive of whether a specific type of degeneration is present.

The neurodegenerative abnormalities may include tissue abnormality or neurological disease such as neurotraumatic brain injury, aneurysm, tremor, spasm, epileptic seizure, tumor, edema, and the like, in or around the brain of the subject. In some instances, multiple comparisons or assessments can be performed on the feature sets and the predictions can be combined by various techniques including simple average, weighted average, majority voting or other similar techniques to diagnose neurodegenerative abnormality.

In addition, one or more actions can be performed if a subject has been identified with a diagnosis of neurodegenerative abnormality based on the prediction model 330. Such predictions may be incorporated into a system that can perform the one or more actions related to both clinical interventions and safety measures to improve health of the subject and to reduce risks. These actions may include, but not limited to, generating alerts, notifying the concerned authorities (e.g., medical staff, relatives and/or the subject) for a complete medical evaluation to confirm the diagnosis of the neurodegenerative abnormality such as brain tumor, stroke, aneurysm, tremor, and the like.

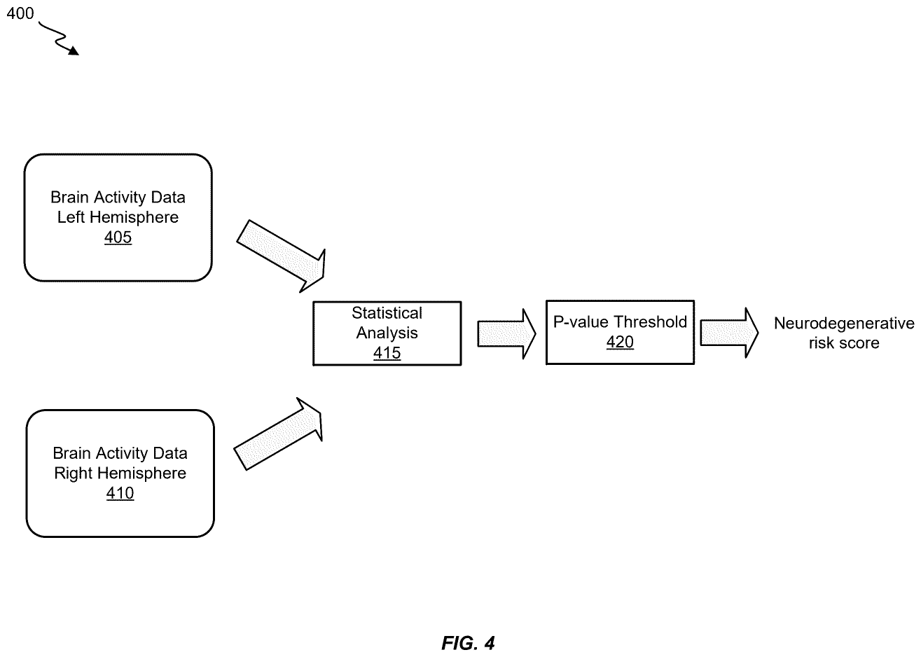

FIG. 4 shows an exemplary implementation of the prediction model 330 of FIG. 3 by using statistical analysis on brain activity data of both hemispheres. Brain activity data of left hemisphere 405 and brain activity data of right hemisphere 410 may be comprised of the feature sets that are extracted using the EEG signals of the left hemisphere and the right hemisphere, respectively (e.g., by using the data pipelines 335a-b of FIG. 3). The feature sets may be computed using different segments or epochs of EEG signals for each of the two hemispheres. These feature sets may represent characteristics of neural activity over time and can be used to quantify differences between the right and the left hemispheres. To analyze asymmetry or abnormality in neural activity between the two hemispheres of the brain, multi-dimensional distributions may be generated based on the extracted feature sets (or brain activity data of left hemisphere 405 and brain activity data of right hemisphere 410). For each hemisphere, a multi-dimensional distribution may be generated by using the associated features (or feature sets). A statistical analysis 415 may be performed on the multi-dimensional distributions to determine whether they stem from the same underlying distribution or if there are significant differences indicative of neurodegenerative abnormalities.

The statistical analysis 415 may utilize one or more statistical tests such as Kolmogorov-Smirnov test, t-test or Wilcoxon signed-rank test. The Kolmogorov-Smirnov test can evaluate whether the multi-dimensional distributions (or cumulative distribution functions) of the two sets of features (one from each hemisphere) differ significantly. Alternatively, methods like the t-test or the Wilcoxon signed-rank test can be employed depending on the nature of the brain activity data (405 and 410) and assumptions about distributional properties. The statistical tests may yield a p-value that quantifies the probability that observed differences between the distributions are due to chance.

To determine the neurodegenerative risk score of the subject, the p-value that is generated using the statistical analysis 415 can be compared to a p-value threshold 420. A smaller value of p-value (e.g., p-value less than 0.05) may be indicative of asymmetry in neural activity across hemispheres and presence of neurodegenerative abnormality.

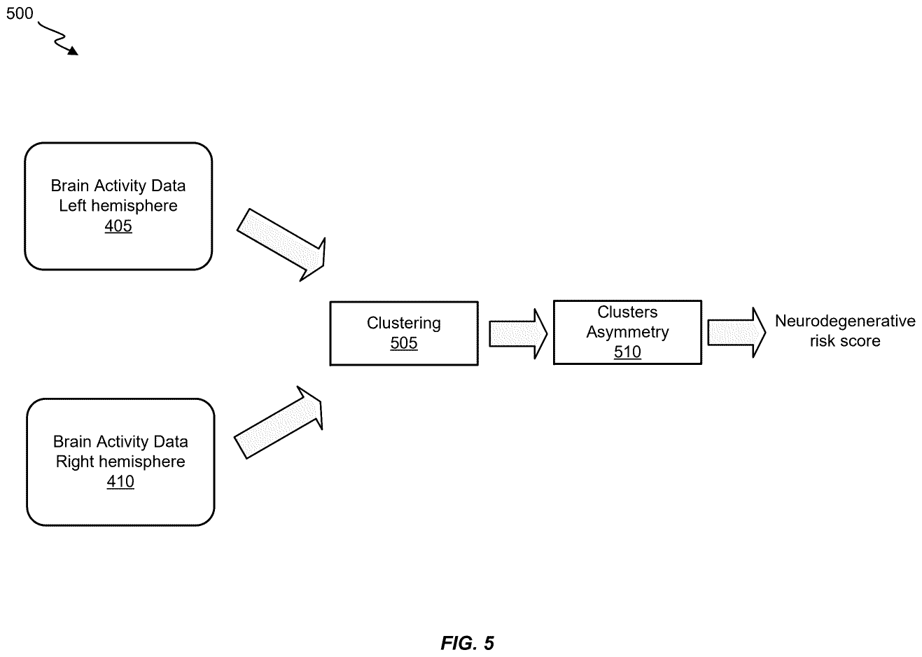

FIG. 5 shows another exemplary implementation of the prediction model 330 of FIG. 3 by leveraging clustering techniques on brain activity data of both hemispheres. When a subject (or healthy subject without neurodegenerative issues) is sleeping or resting, the EEG signals from the right hemisphere and the left hemisphere typically exhibit patterns of synchronization and similar power in different frequency bands. This phenomenon is known as bilateral symmetry in EEG signals during resting or sleeping states. Bilateral symmetry is a characteristic feature of resting or sleep states, reflecting synchronized brain activity between the two hemispheres. In some aspects of the present disclosure, a clustering 505 technique may be applied for determination of asymmetry between the brain activity data of left hemisphere 405 and the brain activity data of right hemisphere 410. The clustering techniques may include but not limited to k-means clustering, hierarchical clustering or Gaussian mixture model (GMM). Each feature set (representing the brain activity data of left hemisphere 405 and right hemisphere 410) corresponding to a given epoch and a given hemisphere may be assigned to a cluster. Clustering 505 may group similar feature vectors together, thereby identifying patterns and similarities within and between the left and right hemispheres.

After the clustering 505 process, a quantification of how closely related or different the neural activity patterns are between the two hemispheres can be performed using the clusters or clustering results. A probabilistic measure or metric of asymmetry can be computed by comparing the distribution of feature vectors assigned to clusters in the left hemisphere with those in the right hemisphere. This comparison may involve statistical metrics such as the Kolmogorov-Smirnov test, which evaluates differences in the distributions of feature vectors. To quantify clusters asymmetry 510, a probability value or score can be derived based on how dissimilar the clusters are between the left and right hemispheres. Higher dissimilarity across clusters of the two hemispheres may be reflected by lower similarity scores or higher probability values (indicating significant differences in distributions). The probability value may correspond to the neurodegenerative risk score. The probability value can then be compared to a predefined probability threshold. The higher neurodegenerative risk score or the probability value greater than the probability threshold, indicates neural dysfunction or pathology and an alert may be generated to inform the subject and/or a caregiver. In the case of healthy subjects (i.e., without neurodegenerative abnormalities) the clustering 505 may reveal that the feature sets extracted from the EEG signals of the left and right hemispheres form cohesive clusters, indicating bilateral symmetry in neural activity patterns.

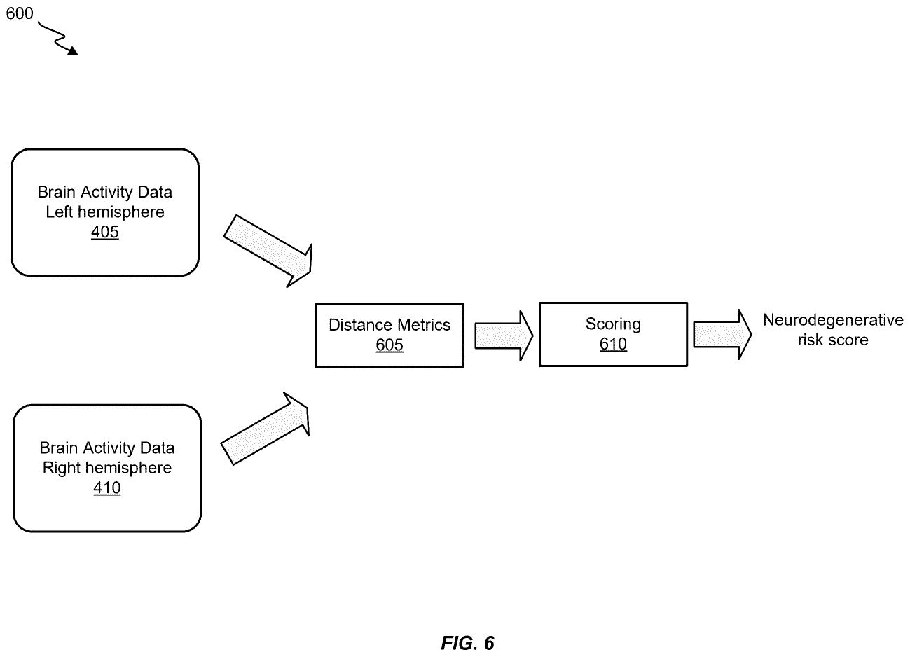

FIG. 6 shows another exemplary implementation of the prediction model 330 of FIG. 3 by utilizing distance metrics to generate the neurodegenerative risk score of the subject. The feature sets (e.g., normalized features, derived features) extracted from the EEG signals of each hemisphere may characterize neural activity, such as power levels across different frequency bands. The feature sets or the brain activity data of left hemisphere 405 and right hemisphere 410 may then be projected into a multi-dimensional space, where each point represents a feature vector from either the left or the right hemisphere.

To assess asymmetry, one or more inter-hemisphere distance metrics 605 may be computed based on the distances between feature vectors from different hemispheres. The one or more inter-hemisphere distance metrics can be obtained by calculating various distance measures, such as Euclidean distance, Mahalanobis distance (covariance between features), or cosine similarity (measure angular difference). These metrics capture the dissimilarity or similarity in feature vectors between corresponding points across hemispheres. Moreover, one or more intra-hemisphere distance metrics 605 can also be computed within each hemisphere, reflecting distances between feature vectors from the same hemisphere. These intra-hemisphere metrics provide a baseline of similarity for comparison against inter-hemisphere distances.

A composite score may be formulated using these distance metrics at scoring 610 module. One approach is to define a score that incorporates both inter-hemisphere distances (reflecting asymmetry) and intra-hemisphere distances (reflecting bilateral similarity). For instance, a ratio or difference between inter-hemisphere and intra-hemisphere distances can quantify the degree of asymmetry, where larger ratios or differences suggesting greater asymmetry between hemispheres and a higher neurodegenerative risk score.

Additionally, statistical techniques such as computing z-scores or normalized distances relative to a reference distribution (e.g., healthy controls) can further refine the assessment of asymmetry. The statistical normalization can account for individual variability and can increase the sensitivity of detecting abnormal asymmetry patterns indicative of neurodegenerative conditions.

Consequently, by comparing the composite score or neurodegenerative risk score against a predefined threshold, clinicians and/or caregivers can determine the presence and severity of asymmetry in neural activity between brain hemispheres. The present disclosure discloses embodiments that leverages quantitative analysis of EEG-derived features and distance metrics within a multi-dimensional space, to identify potential neurodegenerative abnormalities based on distinct patterns of neural activity observed across hemispheres. According to present disclosure, the detection of neurodegenerative abnormalities (e.g., tissue abnormality or neurological disease etc.) and generation of alert may happen in real-time or near real-time.

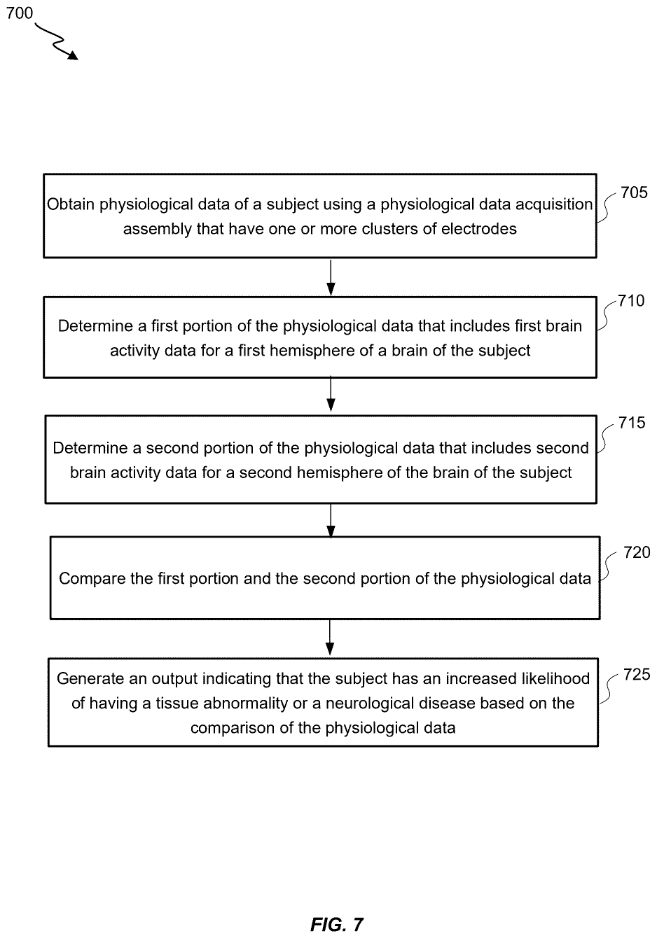

FIG. 7 shows an example flowchart of a system detecting neurodegenerative abnormality in a subject in accordance with some embodiments of the present disclosure. The blocks in flowchart 700 are illustrated in a specific order, while the order can be modified, for example, some blocks may be performed before other, and some blocks may be performed simultaneously. The blocks can be performed by hardware or software or a combination thereof. The process at block 705 may include obtaining physiological data of the subject that was collected by a physiological data acquisition assembly that comprises one or more clusters of electrodes. Each cluster of the one or more clusters of electrodes comprises at least an active electrode. In some instances, the electrodes can be EEG electrodes to record the EEG signals of the left and right hemisphere of the brain of the subject.

A first portion of the physiological data may be determined that includes first brain activity data for a first hemisphere of the brain of the subject, at block 710. A second portion of the physiological data may be determined that includes second brain activity data for a second hemisphere of the brain of the subject, at block 715. The first portion and the second portion of the physiological data corresponding to the first brain activity data and the second brain activity data may include processed EEG signals or feature sets. The feature sets may be obtained by processing the EEG signals using the data pipelines 335a-b. A feature set may be generated by using a segment of EEG signals or an epoch of the EEG signals. The feature set may be comprised of normalized features and/or normalized derived features.

The first portion and the second portion of the physiological data may be compared, at block 720. For example, the feature sets representing the first portion and the second portion of the physiological data may be assessed using a criterion to predict whether there is asymmetry across hemispheres to be indicative of a neurodegenerative abnormality. The feature sets may be projected into a multi-dimensional space and can be assessed using statistical techniques, clustering techniques, or distance metrics. The criterion may identify a threshold for a p-value, probability, score, metric, such that a corresponding subject-specific calculated value can be compared to threshold to predict whether the subject have a neurodegenerative abnormality. Finally, at block 725, an output may be generated based on the comparison of the first portion and the second portion of the physiological data. The output may indicate that the subject has an increased likelihood of having a tissue abnormality or a neurological disease. The output may include presenting the result on the computing device 115 or transmitting the result to another device. The output may include an alert that further investigation of treatment for a neurodegenerative anomaly may be prudent.

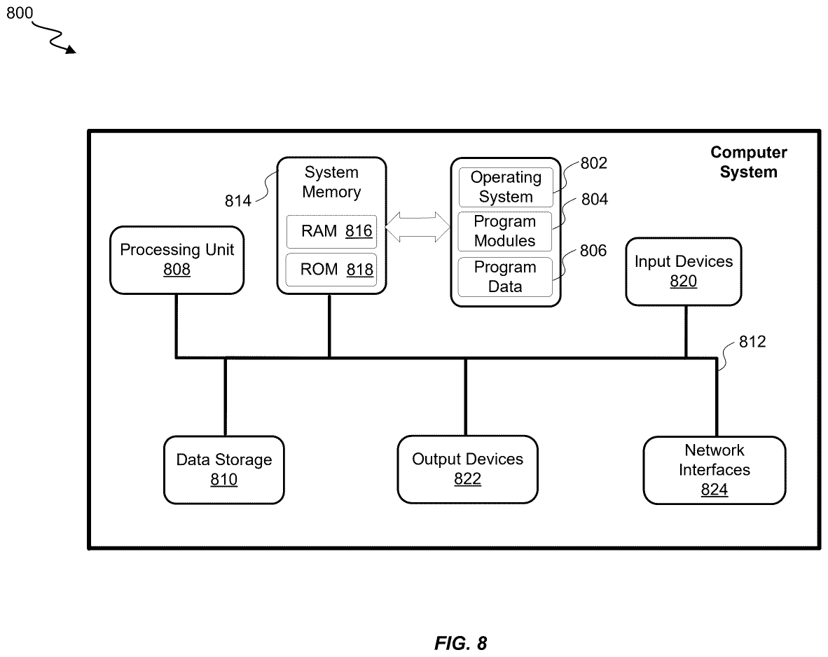

FIG. 8 shows an example illustration of a computer system 800 in which various embodiments of the present disclosure may be implemented. The computer system 800 can be used as the computing device 115 as explained in FIG. 1. For example, the techniques as disclosed above in the present disclosure for detecting the neurodegenerative abnormality using brain activity data can be implemented in computer-executable instructions (e.g., organized in program modules 804). The program modules 804 can include the routines, programs, objects, components, and data structures that perform the tasks and implement the data types for implementing the techniques described above. The functionality described herein can be performed, at least in part, by one or more hardware logic components.

To provide additional context for various aspects thereof, FIG. 8 and the following description are intended to provide a brief, general description of the computer system 800 in which the various aspects can be implemented. While the description above is in the general context of computer-executable instructions that can run on one or more computers, those skilled in the art will recognize that a novel implementation also can be realized in combination with other program modules and/or as a combination of hardware and software. Computer system 800 for implementing various aspects includes a processing unit 808 having one or more processors (also referred to as microprocessors), a computer-readable storage medium (where the medium is any physical device or material on which data can be electronically and/or optically stored and retrieved) such as a data storage 810 unit (computer readable storage medium/media also include magnetic disks, optical disks, solid state drives, external memory systems, and flash memory drives), and a system bus 812. The system bus 812 may provide an interface for system components including, but not limited to, system memory 814, to processing unit 808. Such a system bus 812 can be of any of several types of bus structure that can further interconnect to memory bus (with or without controller), and a peripheral bus (e.g., PCI, PCIe, AGP, LPC, etc.), using any of a variety of commercially available bus architectures.

FIG. 8 shows an example configuration of a typical computer that may be other commercially available microprocessors such as single-processor, multi-processor, single-core units, and multi-core units of processing and/or storage circuits. Moreover, those skilled in the art will appreciate that the novel system and methods can be practiced with other computer system configurations, including minicomputers, mainframe computers, as well as personal computers (e.g., desktop, laptop, tablet PC, etc.), hand-held computing devices, microprocessor-based or programmable consumer electronics, and the like, each of which can be cooperatively coupled to one or more associated devices.

In some aspects, the computer system 800 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example. In some instances, system memory 814 can include computer-readable storage (physical storage) medium such as a volatile memory (e.g. random-access memory (RAM) 816) and a non-volatile memory (e.g., (ROM) 818). A basic Input/output system (BIOS) can be stored in the non-volatile memory and includes the basic routines that facilitate the communication of data and signals between components within the computer system 800, such as during startup. The volatile memory also includes a high-speed RAM such as static RAM for caching data.

By way of example, and not limitation, system memory 814 also may also include program modules 804, which may include client applications, Web browsers, mid-tier applications, relational database management systems (RDBMS), etc., program data 806, and an operating system 802. By way of example, operating system 802 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome OS, and the like) and/or mobile operating systems such as iOS, Windows® Phone, Android OS, BlackBerry® OS, and Palm® OS operating systems. All or portions of operating system 802, program modules 804, and/or program data 806 can also be cached in memory such as the volatile memory and/or non-volatile memory, for example (RAM 816 or ROM 818). It is to be appreciated that the disclosed architecture can be implemented with various commercially available operating systems or combinations of operating systems (e.g., virtual machines).

In some other examples, the computer system 800 may have additional features or functionality. For example, the computer system 800 may also include additional data storage devices (removable /d/ or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer readable instructions, data structures, program modules, or other data.

The system memory 814, and data storage 810 including removable storage, and non-removable storage are all examples of computer storage media. Apart from RAM 816 and ROM 818, computer storage media includes, but is not limited to, EEPROM, flash memory or other memory technology, CD-ROM, digital versatile disks (DVD), or other optical storage, magnetic cassettes, magnetic tape, magnetic disk storage or other magnetic storage devices, or any other non-transmission medium that can be used to store the targeted information and which can be accessed by computer system 800. Moreover, the computer readable media may include computer-executable instructions that, when executed by the processing unit 808, perform various functions and/or operations described herein. The communication media may embody computer readable instructions, data structures, program modules, or other data in a modulated data signal, such as a carrier wave, or other transmission mechanism.

The computer system 800 may also include one or more input devices 820 such as keyboard, mouse, pen, voice input device, touch input device, etc. One or more output devices 822 such as a display, speakers, printers, etc. may also be included. These devices are well known in the art and are not discussed at length here. The computer system 800 may also include one or more network interfaces 824 to establish communication that may allow computer system 800 to communicate with other system or devices, such as over a network. These networks may include wired networks as well as wireless networks. Here, the computer system 800 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game con soles, programmable consumer electronics, network PCs, minicomputers, mainframe computers, distributed computing environments that include any of the above systems or devices, and/or the like. For example, some or all of the components of computer system 800 may be implemented in a cloud computing environment, such that resources and/or services are made available via a computer network for selective use by the user devices.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
obtaining physiological data of a subject that was collected by a physiological data acquisition assembly that comprises one or more clusters of electrodes, wherein each cluster of the one or more clusters of electrodes comprises at least an active electrode;
determining a first portion of the physiological data that includes first brain activity data for a first hemisphere of a brain of the subject;
determining a second portion of the physiological data that includes second brain activity data for a second hemisphere of the brain of the subject;
comparing the first portion and the second portion of the physiological data; and
generating, based on the comparison, an output indicating that the subject has an increased likelihood of having a tissue abnormality or a neurological disease.

2. The computer-implemented method of claim 1, wherein:
the first brain activity data comprises feature sets corresponding to different segments or epochs of EEG signals of the first hemisphere; and
the second brain activity data comprises feature sets corresponding to different segments or epochs of EEG signals of the second hemisphere.

3. The computer-implemented method of claim 1, wherein the comparison of the first portion and the second portion of the physiological data is further comprising:
generating a multi-dimensional distribution for each of the first brain activity data and the second brain activity data; and
comparing the multi-dimensional distribution corresponding to the first brain activity data with the multi-dimensional distribution corresponding to the second brain activity data using a statistical technique.

4. The computer-implemented method of claim 1, wherein the comparison of the first portion and the second portion of the physiological data is further comprising:
performing a clustering technique using the first brain activity data and the second brain activity data;
generating a first set of clusters based on the first brain activity data;
generating a second set of clusters based on the second brain activity data; and
comparing the first set of clusters with the second set of clusters based on a distribution of feature sets of the first brain activity data and distribution of feature sets of the second brain activity data.

5. The computer-implemented method of claim 1, wherein the comparison of the first portion and the second portion of the physiological data is further comprising:
computing an inter-hemisphere distance metric based on the first portion and the second portion of the physiological data;
computing an intra-hemisphere distance metric for each the first portion and the second portion of the physiological data; and
generating a composite score based on the inter-hemisphere distance metric and the intra-hemisphere distance metric for each the first portion and the second portion of the physiological data.

6. The computer-implemented method of claim 1, wherein the neurological disease includes a stroke or aneurysm, or brain tumor.

7. The computer-implemented method of claim 1, further comprising:
predicting a type or location of the tissue abnormality or neurological disease based on the comparison.

8. The computer-implemented method of claim 1, wherein the output includes a p-value, a probability value, or a composite score.

9. A system comprising:
one or more data processors; and
a non-transitory computer readable storage medium containing instructions which, when executed on the one or more data processors, cause the one or more data processors to perform a set of operations including:
accessing physiological data of a subject that was collected by a physiological data acquisition assembly that comprises one or more clusters of electrodes, wherein each cluster of the one or more clusters of electrodes comprises at least an active electrode;
determining a first portion of the physiological data that includes first brain activity data for a first hemisphere of a brain of the subject;
determining a second portion of the physiological data that includes second brain activity data for a second hemisphere of the brain of the subject;
comparing the first portion and the second portion of the physiological data; and
generating, based on the comparison, an output indicating that the subject has an increased likelihood of having a tissue abnormality or a neurological disease.

10. The system of claim 9, wherein the comparison of the first portion and the second portion of the physiological data is further comprising:
performing a clustering technique using the first brain activity data and the second brain activity data;
generating a first set of clusters based on the first brain activity data;
generating a second set of clusters based on the second brain activity data; and
comparing the first set of clusters with the second set of clusters based on a distribution of feature sets of the first brain activity data and distribution of feature sets of the second brain activity data.

11. The system of claim 9, wherein the comparison of the first portion and the second portion of the physiological data is further comprising:
computing an inter-hemisphere distance metric based on the first portion and the second portion of the physiological data;
computing an intra-hemisphere distance metric for each the first portion and the second portion of the physiological data; and
generating a composite score based on the inter-hemisphere distance metric and the intra-hemisphere distance metric for each the first portion and the second portion of the physiological data.

12. The system of claim 9, wherein the physiological data acquisition assembly includes an adhesive film.

13. The system of claim 9, wherein at least of the one or more data processors are within the physiological data acquisition assembly.

14. The system of claim 9, wherein the physiological data acquisition assembly includes a transceiver configured to communicate using a cellular network.

15. The system of claim 9, wherein at least one cluster of the one or more clusters of electrodes comprises a ground electrode.

16. The system of claim 9, wherein at least part of the physiological data acquisition assembly is configured to be reusable across use cases.

17. A computer-program product tangibly embodied in a non-transitory machine-readable storage medium, including instructions configured to cause one or more data processors to perform a set of operations comprising:
obtaining physiological data of a subject that was collected by a physiological data acquisition assembly that comprises physiological data acquisition assembly one or more clusters of electrodes, wherein each cluster of the one or more clusters of electrodes comprises at least an active electrode;
determining a first portion of the physiological data that includes first brain activity data for a first hemisphere of a brain of the subject;
determining a second portion of the physiological data that includes second brain activity data for a second hemisphere of the brain of the subject;
comparing the first portion and the second portion of the physiological data; and
generating, based on the comparison, an output indicating that the subject has an increased likelihood of having a tissue abnormality or a neurological disease.

18. The computer-program product of claim 17, wherein:
the first brain activity data comprises feature sets corresponding to different segments or epochs of EEG signals of the first hemisphere; and
the second brain activity data comprises feature sets corresponding to different segments or epochs of EEG signals of the second hemisphere.

19. The computer-program product of claim 17, wherein the comparison of the first portion and the second portion of the physiological data is further comprising:
generating a multi-dimensional distribution for each of the first brain activity data and the second brain activity data; and
comparing the multi-dimensional distribution corresponding to the first brain activity data with the multi-dimensional distribution corresponding to the second brain activity data using a statistical technique.

20. The computer-program product of claim 17, wherein the comparison of the first portion and the second portion of the physiological data is further comprising:
performing a clustering technique using the first brain activity data and the second brain activity data;
generating a first set of clusters based on the first brain activity data;
generating a second set of clusters based on the second brain activity data; and
comparing the first set of clusters with the second set of clusters based on a distribution of feature sets of the first brain activity data and distribution of feature sets of the second brain activity data.

21. The computer-program product of claim 17, wherein the comparison of the first portion and the second portion of the physiological data is further comprising:
computing an inter-hemisphere distance metric based on the first portion and the second portion of the physiological data;
computing an intra-hemisphere distance metric for each the first portion and the second portion of the physiological data; and
generating a composite score based on the inter-hemisphere distance metric and the intra-hemisphere distance metric for each the first portion and the second portion of the physiological data.

22. The computer-program product of claim 17, wherein the neurological disease includes a stroke, aneurysm, or a brain tumor.
